(19) United States
(12) Patent Application Publication
(10) Pub. No.: US 2002/0107508 A1
(43) Pub. Date: Aug. 8, 2002

(54) Incisionless corneal sculpting technique and devices

(76) Inventor: Daniel R. Burnett, Jacksonville, FL (US)

Correspondence Address: LEARY & ASSOCIATES, 3900 NEWPARK MALL RD., THIRD FLOOR, SUITE 317, NEWARK, CA, 94560, US

(21) Appl. No.: 10/061,428

(22) Filed: Feb. 1, 2002

Related U.S. Application Data
(60) Provisional application No. 60/266,457, filed on Feb. 5, 2001.

(51) Int. Cl. A61B 18/20

(52) U.S. Cl. 606/5; 604/20

(57) ABSTRACT

An apparatus for incisionless corneal sculpting uses a impregnated contact lens with a lateral diffusion shield, a reversible polarity iontophoretic device with an electrode in the form of a conductive contact lens and a laser employing the refractive surgery sculpting protocol, the laser having been tuned to the visible or near-visible wavelength of the dye found in the impregnated contact lens. The method for sculpting the eye is performed by positioning the iontophoretic plate superior to the impregnated contact lens on the surface of the cornea, and setting its polarity to match that of the ionic dye, thereby causing the ionic dye in the contact lens to be driven into the stroma of the cornea. The laser is used to ablate the dyed stroma. After the ablation, the iontophoretic plate is reapplied with reversed polarity and the ionic dye is then extracted from the sculpted cornea.

### CROSS REFERENCE TO OTHER APPLICATIONS

This application claims the benefit of U.S. Provisional Patent Application No. 60/266,457, filed Feb. 5, 2001.

### FIELD OF INVENTION

The present invention relates generally to ocular surgery. More particularly it relates to devices and a method for sculpting the cornea.

### BACKGROUND OF THE INVENTION

Refractive correction has been around for hundreds of years in the form of glasses. Only recently has the technology to change the contour of the cornea arisen. With these techniques, the ophthalmologist can now permanently correct the refractive errors of the human eye.

First came radial keratotomy (RK) in which radial incisions are placed throughout the eye in a wheel and spoke pattern. This relieves the contour of the eye and corrects myopia only. For hyperopia or other problems with eye contour such as astigmatism, this procedure is not much help. Also, the RK procedure carried the inherent risks in making multiple deep incisions into the cornea.

Developments continued and radial keratotomy was replaced by photorefractive keratectomy (PRK). This technique utilized lasers to achieve a much more effective refraction through sculpting the cornea itself. In sculpting the cornea, both the epithelium and stroma of the cornea were obliterated in order to change the overall shape. Ablating the epithelium, though, carried inherent risks in that losing this protective layer frequently resulted in chronic eye conditions such as corneal ulcers and chronic corneal haze.

To counter this side effect, laser in situ keratomileusis (LASIK) was developed. With LASIK, the corneal epithelium is incised and folded back, the stroma is ablated and sculpted, and then the epithelium is repositioned so that it can heal in its native location.

While a major advance over PRK, LASIK still carried inherent risks. Relatively common complications include the loss of the flap of corneal epithelium, a non-healing ulcer in the corneal epithelium, night blindness, and chronic irritation from the epithelial incision. A less frequent, but much more grave, complication is that of enucleation (loss of the eye) as a result of an excessively deep incision into the cornea.

### SUMMARY OF THE INVENTION

The present invention takes the form of an incisionless system for sculpting the cornea. The system has three main parts: an impregnated contact lens, a reversible polarity iontophoretic device and a laser.

The impregnated contact lens involves the impregnation of dye or other ionic substance such as drugs, into a polymer that is formed in the shape of a contact lens. At the lateral edge of the contact lens is a band of dense polymer, plastic or metal forming a lateral diffusion shield, which prevents the lateral diffusion of the impregnated substance.

The reversible polarity iontophoretic device has an iontophoretic plate shaped to perfectly receive the contour of the impregnated contact lens. The iontophoretic plate forms one of the two electrodes of the iontophoretic device. The second electrode is locatable elsewhere on the body. The device is designed such that the user may reverse the polarity of the electrodes. The impregnated contact lens and the iontophoretic plate allow effective, selective targeting of the cornea with little to no exposure of the sclera or other components of the eye.

A laser tuned to the absorption frequency of the ionic substance is then employed using the refractive surgery sculpting protocol to ablate the dyed stroma. Unlike the prior art, which uses the excimer laser and the femtolaser, the present invention uses a laser with a visible wavelength or a near-visible wavelength.

The current technique removes this risk and makes refractive surgery the safest it has ever been. The current technique involves the following steps:

1) positioning the iontophoretic plate superior to the impregnated contact lens on the surface of the cornea, and setting its polarity to match that of the ionic substance;

2) after driving the ionic substance into the stroma, the impregnated contact lens is removed, the cornea is washed and the iontophoretic plate is reapplied in order to drive the ionic substance beyond the epithelial layer;

3) once the ionic substance is selectively located in the stroma of the cornea, a laser tuned to the wavelength of the ionic substance is used to ablate the dyed stroma;

4) and after the ablation, the iontophoretic plate is reapplied with reversed polarity and the ionic substance is then extracted from the sculpted cornea.

This technique has several advantages over LASIK in that the bulk of the complications associated with LASIK are caused by the initial corneal incision. The technique will allow sculpting without the initial incision, thereby drastically decreasing the overall rate of complications.

### BRIEF DESCRIPTION OF THE DRAWINGS

FIG. 1 shows the system for performing non-invasive corneal sculpting.

FIGS. 2A-2B show cross section and front views of the impregnated contact lens with lateral diffusion shield.

FIGS. 3A-3B are part cross section and front views of the reversible polarity iontophoretic device with contact lens-shaped electrode.

FIGS. 4A-4H show the method steps of the non-invasive corneal sculpting technique.

### DETAILED DESCRIPTION

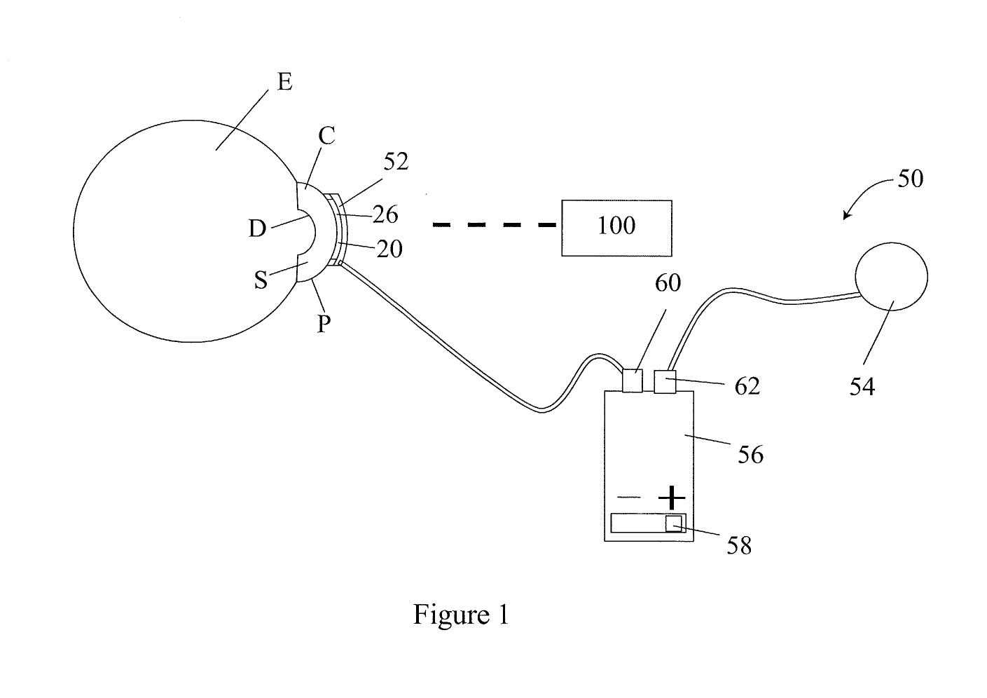

FIG. 1 shows the system for performing non-invasive corneal sculpting. The system 10 is formed of three coordinating devices: an impregnated contact lens 20, a reversible polarity iontophoretic device 50 and a laser 100.

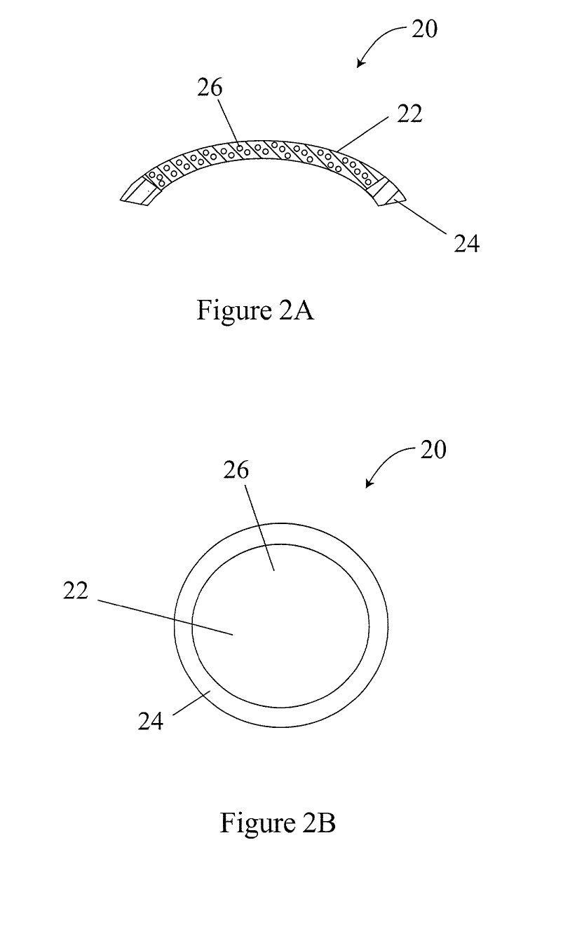

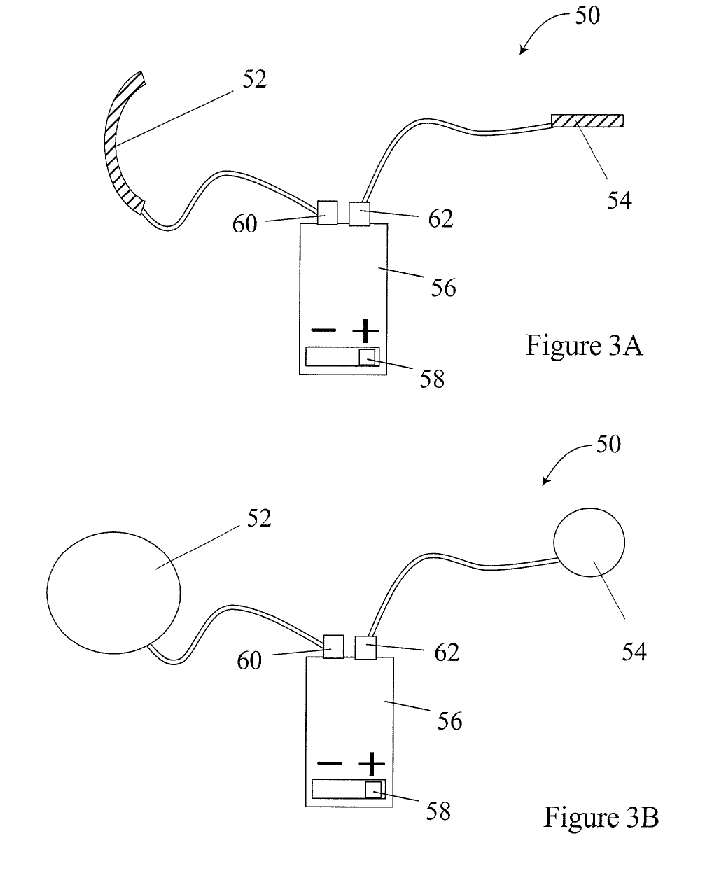

FIG. 2A is a cross section and FIG. 2B is a front view of the impregnated contact lens 20. The contact lens 20 has two main regions, a central polymeric region 22 and a ring 24 at the periphery forming a diffusion barrier. The central region 22 is impregnated with an ionic substance 26 such as dye or drugs capable of diffusing through the epithelial layer and into the stroma of the cornea and having an absorption frequency matched with the wavelength of the laser 100 to be used. Although other types of ionic materials 26 may be used, currently used is a cationic dye, such as Acid Orange 10 with a wavelength of 475 nm, Amarantz with a wavelength of 521 nm, Indigo Carmine with a wavelength of 608 nm and Quinoline Yellow with a wavelength of 412 nm. The diffusion barrier 24 is formed by a band of material that inhibits the ionic substance 26 from passing therethrough. The diffusion barrier 24 may be formed of a different and/or denser polymeric material, plastic or metal, thereby creating a diffusion shield to inhibit lateral diffusion of the ionic substance 26. The impregnated contact lens 20 has been designed to conduct a current and is used with the reversible polarity iontophoretic device 50 shown in FIGS. 3A and 3B.

(Do You Have Specific Materials/Densities for the Polymeric Material of the Contact Lens?)

The reversible polarity iontophoretic device 50 has three main components: a pair of opposing electrodes 52, 54, a battery, outlet connection or other power source 56 and a switch 58 to reverse the polarity of the poles 60, 62. One of the electrodes is an iontophoretic plate 52 having a concave surface sized and configured to receive the impregnated contact lens 20. If desired, the iontophoretic plate 52 may be contact lens shaped also. The iontophoretic plate 52 is connected to the first pole 60 of the power source 56. The other electrode 54 is designed to attach elsewhere on the patient's body, such as the head or neck. The other electrode 54 is connected to the second pole 62 of the power source 56. The power source 56 may then be used to generate the required current.

(Do You Have Data on What Voltage/Current is Appropriate?)

When the iontophoretic plate 52 is placed over the impregnated contact lens 20, the switch 58 may be used to reverse the polarity of the poles 60, 62 and the corresponding electrodes 52, 54. The polarity controls whether the ionic substance 26 is attracted to or repelled from the iontophoretic plate 52, thereby driving the ionic substance 26 from the impregnated contact lens 20 into the eye or out of the eye.

A laser 100 is then used to ablate tissue from the cornea, thereby sculpting the shape thereof. The laser 100 is chosen to match the absorption wavelength of the ionic substance 26. Although other wavelength combinations could be used, currently visible or near-visible wavelengths are preferred. For example, an argon ion laser produces light at 488 and 514 nm and would be optimal for use with either the Acid Orange 10 or the Amarantz dyes. A helium cadmium laser produces light at 325 and 442 nm and would be optimal for use with the Quinoline Yellow dye. Other options include using an infrared laser with protoporphyrin chromphores dyes or an ultraviolet (UV) laser with a Uv absorbing dye.

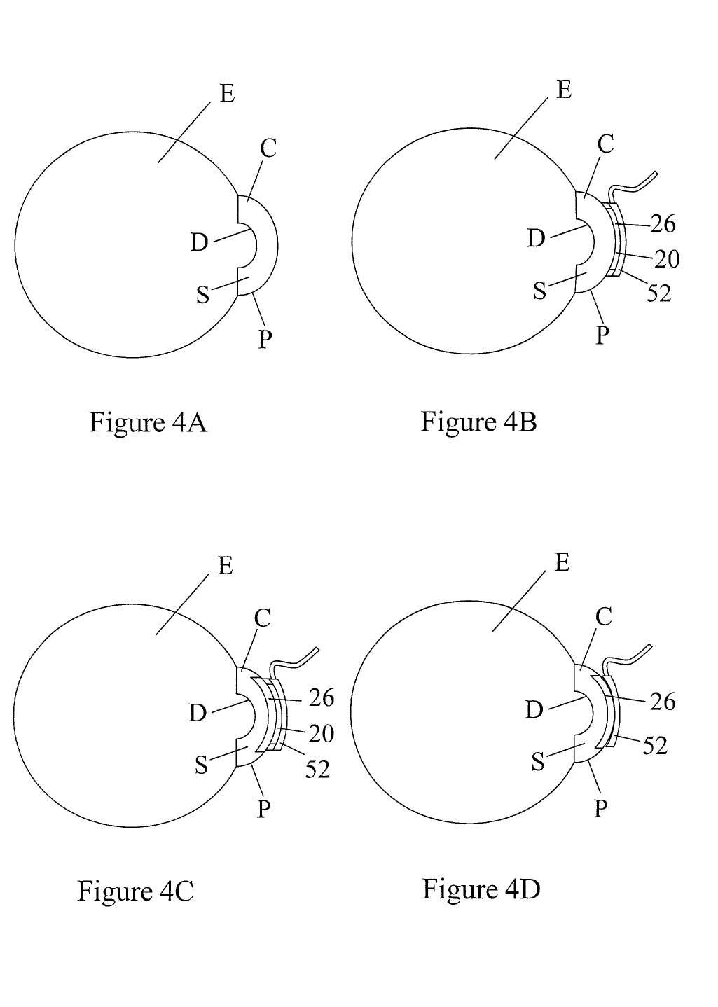

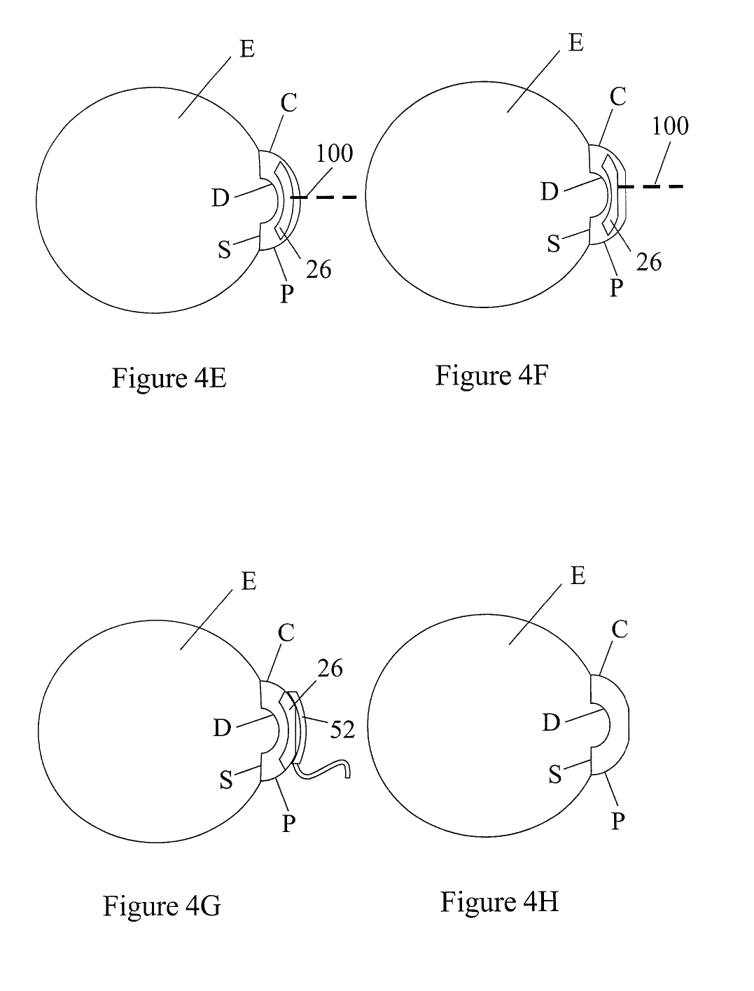

FIGS. 4A-4H show the non-invasive corneal sculpting method. The method is begun by applying medicated drops to the epithelium P of the cornea C to decrease sensation and increase permeability to the ionic substance 26. The impregnated contact lens 20 with lateral diffusion shield 24 is placed on the epithelium P of the cornea C of the eye E to be corrected. The iontophoretic plate 52 of the reversible polarity iontophoretic device 50 is then placed over the impregnated contact lens 20, as seen in FIG. 4B. The second electrode 54 is placed elsewhere on the body of the patient, such as the head or neck. A voltage is applied to the electrodes 52, 54 with the polarity of the iontophoretic plate 52 matching the ionic substance 26 in the impregnated contact lens 20, thereby forcing the ionic substance 26 from the impregnated contact lens 20 into the eye E, as seen in FIG. 4C. Next, the outside of the cornea C is washed free of the ionic substance 26 and the iontophoretic plate 52 is reapplied, without the impregnated lens 20, to drive the ionic substance 26 beyond the epithelial layer P of the cornea C, but above the endothelial layer D, as seen in FIG. 4D. Once the ionic substance 26 is selectively located in the stromal layer S of the cornea C, the iontophoretic plate 52 is removed from the eye E, as seen in FIG. 4E. A laser 100 tuned to the absorption wavelength of the ionic substance 26 is used to sculpt the cornea C using the standard laser refractive surgery protocol as seen in FIGS. 4E and 4F. The level of excitation is selected to be low enough that the tissue is not vaporized. Instead, the tissue is damaged and the collagen is denatured, such that it will be reabsorbed by the cornea C. As seen in FIG. 4G, the iontophoretic plate 52 is then reapplied with its polarity reversed in order to draw the ionic substance 26 out of the cornea C due to the opposite polarity attracting the ionic substance 26. Once the eye E is free of the ionic substance 26, as seen in FIG. 4H, the eye E is allowed to heal and the damaged stromal tissue S resorbs over the course of one to two weeks during which sight improves.

(What Kind of Drops are Added to the Eye?)

Many features have been listed with particular configurations, options, and embodiments. Any one or more of the features described may be added to or combined with any of the other embodiments or other standard devices to create alternate combinations and embodiments.

Although the examples given include many specificities, they are intended as illustrative of only one possible embodiment of the invention. Other embodiments and modifications will, no doubt, occur to those skilled in the art. Thus, the examples given should only be interpreted as illustrations of some of the preferred embodiments of the invention, and the full scope of the invention should be determined by the appended claims and their legal equivalents.

What is claimed is:

1. An iontophoretic device for delivery of an ionic substance to an eye of a patient, the iontophoretic device comprising:
a power source having a first pole and a second pole,
a first electrode having a concave surface sized and configured to receive a contact lens, said first electrode connected to said first pole,
a second electrode sized and configured to be locatable on a body of the patient, said second electrode connected to said second pole,
and a switch for reversing the polarity of said first and second poles of said power source.

2. A contact lens, comprising:
a contact lens body having a concave shape sized and configured to fit over an eye of a patient, said body including:
a central region impregnated with an ionic substance,
a diffusion barrier around a periphery of said central region, said diffusion barrier formed of a material that inhibits said ionic substance from passing therethrough.

3. The contact lens of claim 2, wherein said central region is formed of a polymer.

4. The contact lens of claim 3, wherein said diffusion barrier is formed of a polymer denser than said polymer of said central region.

5. The contact lens of claim 2, wherein said diffusion barrier is formed of plastic.

6. The contact lens of claim 3, wherein said diffusion barrier is formed of metal.

7. The contact lens of claim 2, wherein said ionic substance is a dye.

8. The contact lens of claim 2, wherein said ionic substance is a cationic dye.

9. The system of claim 2, wherein said ionic substance is a drug.

10. The contact lens of claim 2, wherein said contact lens is electrically conductive.

11. A system for sculpting a cornea of an eye of a patient, the system comprising:
a contact lens impregnated with an ionic substance having an absorption wavelength, said contact lens sized and configured to fit over the cornea,
an iontophoretic device with an iontophoretic plate, said iontophoretic plate sized and configured to fit over said contact lens,
and a laser tuned to the absorption wavelength of said ionic substance.

12. The system of claim 11, wherein said ionic substance is a dye.

13. The system of claim 11, wherein said ionic substance is a cationic dye.

14. The system of claim 11, wherein said ionic substance is a drug.

15. The system of claim 11, wherein said con tact lens comprises a central region impregnated with said ionic substance and a diffusion barrier around a periphery of said central region, said diffusion barrier formed of a material that inhibits said ionic substance from passing therethrough.

16. The system of claim 11, wherein said contact lens has a central portion and a periphery and wherein said periphery is formed of a denser material than said central portion.

17. The system of claim 16, wherein said denser material forms a diffusion barrier around said periphery of said contact lens.

18. The system of claim 11, wherein said contact lens is electrically conductive.

19. The system of claim 11, wherein said iontophoretic plate acts as a first electrode and wherein said iontophoretic device further comprises a second electrode and a power source having two poles with opposing polarity, and wherein each of said electrodes are connected to one of said two poles.

20. The system of claim 19, further comprising a switch controlling the polarity of the two poles of the power source.

21. The system of claim 11, wherein said laser has a wavelength in the visible spectrum.

22. The system of claim 11, wherein said laser is an argon ion laser.

23. The system of claim 11, wherein said laser is a helium cadmium laser.

24. A system for sculpting a cornea of an eye of a patient, the system comprising:
a contact lens body having a concave shape sized and configured to fit over the eye, said body including:
a central region impregnated with an ionic substance having an absorption wavelength,
a diffusion barrier around a periphery of said central region, said diffusion barrier formed of a material that inhibits said ionic substance from passing therethrough,
an iontophoretic device including:
a power source having a first pole and a second pole,
a first electrode having a concave surface sized and configured to receive a contact lens, said first electrode connected to said first pole,
a second electrode sized and configured to be locatable on a body of the patient, said second electrode connected to said second pole,
and a switch for reversing the polarity of said first and second poles of said power source,
and a laser tuned to the absorption wavelength of said ionic substance.

25. The system of claim 24, wherein said wavelength is visible or near-visible.

26. The system of claim 24, wherein said ionic substance is a cationic dye.

27. The system of claim 24, wherein said central portion is formed of a first polymer, and wherein said diffusion barrier is formed of a second polymer and wherein said second polymer is denser than said first polymer.

28. A method of sculpting a cornea of an eye of a patient, the method comprising the steps of:
(a) placing a contact lens impregnated with an ionic substance on an epithelium of the eye;
(b) placing an iontophoretic plate over the impregnated contact lens;
(c) applying voltage to the iontophoretic plate with a polarity matching a charge of the ionic substance, thereby forcing the ionic substance from the contact lens into the eye;
(d) removing the iontophoretic plate and the contact lens;
(e) sculpting the cornea using a laser tuned to an absorption wavelength of said ionic substance;
(f) and allowing the eye to heal.

29. The method of claim 28, wherein step (e) is performed with a laser having a visible wavelength.

30. The method of claim 28, wherein step (e) is performed with a laser having a near-visible wavelength.

31. The method of claim 28, further comprising the steps of:
(g) reapplying the iontophoretic plate to the eye;
(h) applying voltage to the iontophoretic plate, thereby driving the dye beyond the epithelium;
(i) and removing the iontophoretic plate.

32. The method of claim 28, further comprising the steps of:
(g) reapplying the iontophoretic plate to the eye;
(h) and charging the iontophoretic plate with a reverse polarity, thereby drawing the ionic substance out of the eye.

33. The method of claim 28, further comprising the steps of:
(g) applying drops to an epithelium of the cornea, thereby decreasing sensation and increasing permeability;
(h) and placing a second electrode on another part of the patient.

34. The method of claim 28, further comprising the steps of:
(g) washing the cornea free any excess of the ionic substance.

35. A method of sculpting a cornea of an eye of a patient, the method comprising the steps of:
(a) applying drops to an epithelium of the cornea, thereby decreasing sensation and increasing permeability;
(b) placing a contact lens impregnated with an ionic substance on the epithelium;
(c) placing an iontophoretic plate over the impregnated contact lens;
(d) placing a second electrode on another part of the patient;
(e) applying voltage to the iontophoretic plate with a polarity matching a charge of the ionic substance, thereby forcing the ionic substance from the contact lens into the eye;
(f) removing the iontophoretic plate and the contact lens;
(g) washing the cornea free of any excess of the ionic substance;
(h) reapplying the iontophoretic plate to the eye;
(i) applying voltage to the iontophoretic plate, thereby driving the dye beyond the epithelium;
(j) removing the iontophoretic plate;
(k) sculpting the cornea using a visible wavelength laser tuned to an absorption wavelength of said ionic substance;
(l) reapplying the iontophoretic plate to the eye;
(m) charging the iontophoretic plate with a reverse polarity, thereby drawing the ionic substance out of the eye;
(n) removing the iontophoretic plate;
(o) washing away any residual ionic substance;
(p) and allowing the eye to heal.
